(19) United States
(12) Patent Application Publication
(10) Pub. No.: US 2023/0310303 A1
(43) Pub. Date: Oct. 5, 2023

(54) SILICONE/HYDROCARBON COSMETIC COMPOSITIONS

(71) Applicant: L'ORÉAL, Paris (FR)

(72) Inventor: Tsang-Min HUANG, Scotch Plains, NJ (US)

(73) Assignee: L'ORÉAL, Paris (FR)

(21) Appl. No.: 17/709,585

(22) Filed: Mar. 31, 2022

(51) Int. Cl. A61K 8/90; A61K 8/81; A61K 8/891; A61K 8/25; A61Q 1/04

(52) U.S. Cl. CPC A61K 8/90; A61K 8/8117; A61K 8/8111; A61K 8/891; A61K 8/25; A61K 2800/612; A61K 2800/48; A61K 2800/524; A61K 2800/436; A61K 2800/594; A61Q 1/04

(57) ABSTRACT

Cosmetic compositions may include a first phase that includes at least about 8% by weight of the composition of a thermoplastic block copolymer comprising styrenated blocks, and at least one hydrocarbon oil, and a second phase that includes at least 20% by weight of the composition of a dimethicone. To provide the necessary stability to such systems, the compositions utilize both a hectorite and silica silylate, where the hectorite is disteardimonium hectorite, stearalkonium hectorite, or both.

### TECHNICAL FIELD

The present disclosure is drawn to cosmetic compositions, and in particular, silicone/hydrocarbon cosmetic lip compositions that also contain a styrenic block copolymer.

### BACKGROUND

Styrenic block copolymer (SBC)/silicone systems are commonly used in conventional lip gloss products. Due to the nature of SBC chemistry, the usage levels are low to maintain good application and reduce tack. Increasing usage levels results in SBC/silicone systems that are not table at elevated temperature, leading to the syneresis of silicone and the settling of any pigment, filler, or pearlescent agent.

Thus, a cosmetic system that allows for increased usage levels of an SBC while maintaining stability and preventing syneresis and/or settling is useful and desirable.

### BRIEF SUMMARY

Disclosed is a composition can provides such improved stability. The cosmetic compositions, such as those for application to lips, may include a first phase that includes at least about 8% by weight of a thermoplastic block copolymer comprising styrenated blocks, and at least one hydrocarbon oil. The composition may include a second phase that includes at least 20% of a viscous silicone such as a dimethicone. To provide the necessary stability to such systems, the compositions utilize a filler that includes both a hectorite and silica silylate, where the hectorite is disteardimonium hectorite, stearalkonium hectorite, or both.

In some embodiments, the dimethicone may include a plurality of dimethicones. In some embodiments, the plurality of dimethicones may include a first dimethicone having a molecular weight less than 1,500 g/mol (such as cetyl dimethicone, with a molecular weight of 1,000 g/mol), and a second dimethicone having a molecular weight greater than 2,000 g/mol (such as trimethylsiloxyphenyl dimethicone with a molecular weight of 3,000 g/mol). In some embodiments, first dimethicone may be present at less than 10% by weight of the composition, and a second dimethicone that is present at more than 15% by weight of the composition. In some embodiments, the weighted average of the molecular weights of the plurality of dimethicones is at least 7000 g/mol. In some embodiments, the dimethicone may include a first dimethicone that is a polyalkyl dimethicone, and a second dimethicone that contains at least one aryl group.

In some embodiments, the thermoplastic block copolymer may be present in an amount of at least 10% by weight of the composition.

In some embodiments, the composition may include at least 0.5% by weight of the hectorite. In some preferred embodiments, the composition may include at least 1% by weight of the hectorite. In some more preferred embodiments, the composition may include 1.5% to 2.5% by weight of the hectorite.

In some embodiments, the hydrocarbon oil comprises a saturated or unsaturated polyolefin, such as hydrogenated polyisobutene.

In some embodiments, the composition may include a pigment, a pearlescent agent, or a combination thereof. In some embodiments, the composition may include a thickener, an olfactive sensorial agent, a solvent, a preservative, a butter, an ester, or a combination thereof. In some embodiments, the composition may include a fatty compound having a melting point in the range of 30° C. to 50° C., such as Butyrospermum Parkii (Shea) Butter and/or Bis-diglyceryl polyacyladipate-2.

In some embodiments, a storage modulus at a temperature of 55° C. divided by a storage modulus at a temperature of 25° C. is greater than or equal to 0.08.

In some embodiments, a ratio of the amount of hectorite to the amount of silica silylate is between 0.25:1 and 1:1.

In some embodiments, a method is disclosed for providing a high gloss, low tack lip composition. The method includes forming a double phase product on the lips from a single product by applying a composition according to claim 1 and breaking the composition such that it forms a first layer comprising the thermoplastic block copolymer in contact with the lips and a second layer comprising the dimethicone on top of the first layer.

### BRIEF DESCRIPTION OF DRAWINGS

FIG. 1 is a representation of a composition as seen under a microscope.

FIG. 2 is a graph illustrating storage modulus values for two different compositions at different temperatures.

FIG. 3 is a graph illustrating storage modulus values for eight different compositions at different temperatures.

FIG. 4 is an exaggerated illustration of a subject's lips with the first phase and the second phase being applied in separate layers.

### DETAILED DESCRIPTION

As used herein, the terms “comprising,” “having,” and “including” are used in their open, non-limiting sense.

The terms “a,” “an,” and “the” are understood to encompass the plural as well as the singular. Thus, the term “a mixture thereof” also relates to “mixtures thereof.” Throughout the disclosure, the term “a mixture thereof” is used, following a list of elements as shown in the following example where letters A-F represent the elements: “one or more elements selected from the group consisting of A, B, C, D, E, F, and a mixture thereof.” The term, “a mixture thereof” does not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.”

The expression “one or more” means “at least one” and thus includes individual components as well as mixtures/combinations.

The term “free” or “completely free of (a component)” as defined herein means that the systems or compositions do not contain the component in any measurable degree by standard means.

The term “substantially free of (a component)” as defined herein means that the systems or compositions contain no appreciable amount of the component, for example, no more than about 1% by weight, or no more than about 0.5% by weight, or no more than about 0.3% by weight, such as no more than about 0.1% by weight, based on the weight of the system or composition comprising the system and/or the oxidizing composition according to embodiments of the disclosure.

Other than in the operating examples, or where otherwise indicated, all numbers expressing quantities of ingredients and/or reaction conditions may be modified in all instances by the term “about,” meaning within +/−5% of the indicated number.

As used herein, all ranges provided are meant to include every specific range within, and combination of sub ranges between, the given ranges. Thus, a range from 1-5, includes specifically 1, 2, 3, 4 and 5, as well as sub-ranges such as 2-5, 3-5, 2-3, 2-4, 1-4, etc.

All components and elements positively set forth in this disclosure can be negatively excluded from the claims. In other words, the cosmetic compositions of the instant disclosure can be free or substantially free of all components and elements positively recited throughout the instant disclosure.

Disclosed is a cosmetic composition, which may be a composition for application to lips. The composition may include a first phase that includes: (1) at least about 8% by weight of the composition of a thermoplastic block copolymer comprising styrenated blocks; and (2) at least one hydrocarbon oil. The composition may include a second phase that has at least 20% by weight of the composition of a viscous silicone, which may be a dimethicone. The composition may include a filler, where the filler includes both a hectorite and a silica silylate.

In some embodiments, these compositions are in the form of a gel.

Preferably, these compositions may be free of water. In some embodiments, these compositions may be substantially free of water.

Preferably, these compositions may be free of an emulsifier. In some embodiments, these compositions may be substantially free of an emulsifier.

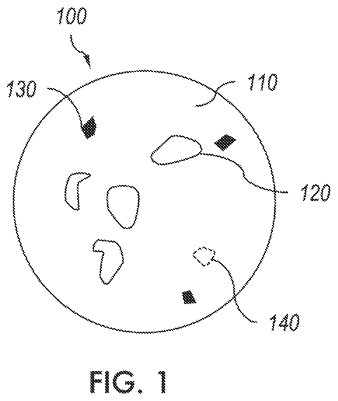

Referring briefly to FIG. 1, a simplified illustration of the composition can be seen. In FIG. 1, the composition 100 is seen as comprising a first phase 110, an oil phase containing a thermoplastic block copolymer comprising styrenated block and a hydrocarbon oil. The composition also includes a second phase 120 that comprises the silicones. The second phase is dispersed in/encapsulated by the first phase. It can be seen that the second phase is not forming spherical droplets as one would expect in, e.g., a water-in-oil emulsion. Rather, these are seen as discrete, non-spherical volumes contained within the first phase when viewed under a microscope. The filler 130 is present to stabilize the composition, and other components, including, e.g., particulates 140 such as pigments may be present.

Thermoplastic Block Copolymer Comprising Styrenated Blocks

In some embodiments, the composition may include one or more thermoplastic block copolymers comprising styrenated blocks. In some embodiments, the one or more thermoplastic block copolymers may consist of a single thermoplastic block copolymer. In some embodiments, the one or more thermoplastic block copolymers may consist of a plurality of thermoplastic block copolymers.

The block copolymers of the present invention are characterized by the presence of at least one “hard” segment, and at least one “soft” segment. Aside from their compositional nature, the hard and soft segments of the block copolymers of the present invention may be defined in terms of their respective glass transition temperatures, T_{g}. The hard segment may have a T_{g} of 50° C. or more, whereas the soft segment may have a T_{g} of 20° C. or less. The T_{g} for the hard block can range from 50° C. to 150° C. The T_{g} for the soft block can range from can range from −150° C. to 20° C.

Block copolymers useful in compositions of the present invention may be thermoplastic elastomers. The hard segments of the thermoplastic elastomer typically comprise vinyl monomers in varying amounts. Examples of suitable vinyl monomers include, but are not limited to, styrene as well as other optional monomers including methacrylate, acrylate, vinyl ester, vinyl ether, vinyl acetate, and the like.

The soft segments of the thermoplastic elastomer comprise olefin polymers and/or copolymers which may be saturated, unsaturated, or combinations thereof. Suitable olefin copolymers may include, but are not limited to, ethylene/propylene copolymers, ethylene/butylene copolymers, propylene/butylene copolymers, polybutylene, polyisoprene, polymers of hydrogenated butanes and isoprenes, and mixtures thereof.

Thermoplastic elastomers useful in the present invention are block copolymers e.g., di-block, tri-block, multi-block, radial and star block copolymers, and mixtures and blends thereof. A di-block thermoplastic elastomer is usually defined as an A-B type or a hard segment (A) followed by a soft segment (B) in sequence. A tri-block is usually defined as an A-B-A type copolymer or a ratio of one hard, one soft, and one hard segment. Multi-block or radial block or star block thermoplastic elastomers usually contain any combination of hard and soft segments, provided that the elastomers possess both hard and soft characteristics.

In some embodiments, the thermoplastic elastomer of the present invention may be chosen from the class of KRATON rubbers (Kraton Corporation of Houston, Tex.) or from similar thermoplastic elastomers. KRATON rubbers are thermoplastic elastomers in which the polymer chains comprise a di-block, tri-block, multi-block or radial or star block configuration or numerous mixtures thereof. The KRATON tri-block rubbers have polystyrene (hard) segments on each end of a rubber (soft) segment, while the KRATON di-block rubbers have a polystyrene (hard) segment attached to a rubber (soft) segment. The KRATON radial or star configuration may be a four-point or other multipoint star made of rubber with a polystyrene segment attached to each end of a rubber segment. The configuration of each of the KRATON rubbers forms separate polystyrene and rubber domains.

Each molecule of KRATON rubber is said to comprise block segments of styrene monomer units and rubber monomer and/or co-monomer units. The most common structure for the KRATON triblock copolymer is the linear A-B-A block type styrene-butadiene-styrene, styrene-isoprene-styrene, styrene-ethylenepropylene-styrene, or styrene-ethylenebutylene-styrene. The KRATON di-block is preferably the AB block type such as styrene-ethylenepropylene, styrene-ethylenebutylene, styrene-butadiene, or styrene-isoprene. The KRATON rubber configuration is well known in the art and any block copolymer elastomer with a similar configuration is within the practice of the invention. Other block copolymers are sold under the tradename Septon (which represent elastomers known as SEEPS, sold by Kurary, Co., Ltd) and those sold by ExxonMobil Chemical under the tradename VECTOR.

Other thermoplastic elastomers useful in the present invention include those block copolymer elastomers comprising a styrene-butylene/ethylene-styrene copolymer (tri-block), an ethylene/propylene-styrene copolymer (radial or star block) or a mixture or blend of the two. (Some manufacturers refer to block copolymers as hydrogenated block copolymers, e.g. hydrogenated styrene-butylene/ethylene-styrene copolymer (tri-block)).

Compositions of the present invention include at least one block copolymer, e.g., diblock, triblock, multiblock or radial block copolymers, and mixtures thereof. The at least one block copolymer comprises at least one styrene block and may further comprise at least one block comprising units chosen from butadiene, ethylene, propylene, butylene and isoprene or a mixture thereof.

Diblock copolymers that may be mentioned include but are not limited to styrene/ethylene-propylene copolymers (comprising a styrene block and a block obtained from ethylene and propylene), styrene/ethylene-butylene copolymers, styrene-ethylene/butadiene copolymers, styrene/butadiene copolymers and styrene/isoprene copolymers.

In certain notable embodiments, the block copolymer includes styrene blocks and one or more blocks selected from: butadiene blocks, isoprene blocks, and ethylene-butylene blocks. In other embodiments, the block copolymer includes (1) styrene blocks and butadiene blocks; or (2) styrene and ethylene-butadiene blocks; or (3) styrene and isoprene blocks.

Triblock copolymers that may be mentioned include but are not limited to styrene/ethylene-propylene/styrene copolymers, styrene/ethylene-butylene/styrene copolymers, styrene/ethylene-butadiene/styrene copolymers, styrene/isoprene/styrene copolymers and styrene/butadiene/styrene copolymers. For instance, triblock polymers sold under the names KRATON G1650, KRATON G1652, KRATON D1101, KRATON D1102 and KRATON, commercially available from the company Kraton Polymers.

For instance, a mixture of a diblock copolymer and of a triblock copolymer may be used as block copolymer. According to at least one embodiment, the diblock copolymer and the triblock copolymer may be chosen from block copolymers comprising at least one styrene block and at least one block comprising units chosen from butadiene, ethylene, propylene, butylene and isoprene. In one embodiment, the block copolymer has from about 50% to about 90% triblock and from about 10% to about 50% diblock.

Examples of suitable block copolymers include Kraton G series such as Kraton G1701 (INCI name of Hydrogenated Styrene/Isoprene Copolymer) and KRATON G1657 (HYDROGENATED STYRENE/BUTADIENE COPOLYMER). KRATON G1657 (styrene-ethylene/butylene-styrene triblock and 30% styrene-ethylene-butylene diblock); INCI: HYDROGENATED STYRENE/BUTADIENE COPOLYMER; a mixture of 70% styrene-ethylene-butylene triblock) is particularly notable.

It is preferred that the styrene content of the block copolymer be less than 30% by weight, preferably less than 25% by weight, and more preferably less than 20% by weight, and more preferably from about 5% to about 15%, based on the weight of the block copolymer. This is because of the tendency of block copolymers having a styrene content of greater than 30% by weight to harden/gel in conventional carrier systems.

In some embodiments, the thermoplastic block copolymer(s) comprising styrenated blocks may be present in the cosmetic composition in a total amount ranging from, for example, about 8% to about 30% by weight; such as from about 9% to about 25% by weight; or such as from about 10% to about 20%, based on the weight of the composition.

In some embodiments, the thermoplastic block copolymer is present in the first phase in an amount of at least 10% by weight of the composition.

Hydrocarbon Oil

In some embodiments, the composition may include one or more hydrocarbon oils. In some embodiments, the composition may include a plurality of hydrocarbon oils. In some embodiments, each hydrocarbon oil is a non-volatile oil.

The term “hydrocarbon oil” is understood to mean an oil predominantly comprising carbon and hydrogen atoms, and optionally ester, ether, fluoro, carboxylic acid and/or alcohol groups. In some embodiments, the hydrocarbon oil only contains carbon and hydrogen atoms.

The term “non-volatile oil” is understood to mean an oil which remains on the skin or a keratinous fibre at ambient temperature (20-25° C.) and atmospheric pressure for at least several hours, and which in particular has a vapour pressure of less than 10⁻³ mmHg (0.13 Pa).

Mention may be made, as examples of non-volatile hydrocarbon oils which can be used in the invention, of:
hydrocarbon oils of vegetable origin, such as triglycerides of fatty acids having from 4 to 24 carbon atoms, such as triglycerides of caprylic/capric acids, such as those sold by Stearinerie Dubois or those sold under the names Miglyol 810®, 812® and 818® by Dynamit Nobel; triglycerides of branched C₁₈-C₃₆ fatty acids and of glycerol, such as that sold under the name DUB TGI 24® by Stearinerie Dubois (INCI name C₁₈₋₃₆ Acid Triglyceride);
linear or branched hydrocarbons of mineral or synthetic origin, such as liquid paraffins and their derivatives, petrolatum, polydecenes, polybutenes, hydrogenated polyisobutene, such as Parleam, or squalane;
synthetic ethers having from 10 to 40 carbon atoms, such as dicaprylyl ether;
synthetic esters, in particular of fatty acids isononyl isononanoate, isopropyl myristate, isopropyl palmitate, C₁₂ to C₁₅ alkyl benzoate, hexyl laurate, diisopropyl adipate, 2-ethylhexyl palmitate, 2-octyldodecyl stearate, 2-octyldodecyl erucate, isostearyl isostearate, diisostearyl malate or tridecyl trimellitate;
fatty alcohols which are liquid at ambient temperature, comprising a branched and/or unsaturated carbon chain having from 12 to 26 carbon atoms, such as octyldodecanol, isostearyl alcohol, 2-butyloctanol, 2-hexyldecanol, 2-undecylpentadecanol or oleyl alcohol;
higher fatty acids, such as oleic acid, linoleic acid or linolenic acid;
carbonates, such as dicaprylyl carbonate or propylene carbonate;
acetates; and
citrates.

In some embodiments, the hydrocarbon oil comprises a saturated or unsaturated polyolefin. In some embodiments, the saturated or unsaturated polyolefin is hydrogenated polyisobutene.

In some embodiments, the hydrocarbon oil(s) may be present in the cosmetic composition in a total amount ranging from, for example, about 5% to about 70% by weight; such as from about 10% to about 60% by weight; or such as from about 20% to about 50%, based on the weight of the composition.

In some embodiments, the hydrocarbon oil(s) may include at least one non-polar oil, and at least one polar oil.

Non-Polar Oil

In certain embodiments, the non-polar oil has a partition coefficient (log P) value of greater than 15, such as greater than about 20. According to certain other embodiments the non-polar oil is selected from an alkane, an olefin (alkene) such as a polyolefin, and combinations thereof. Suitable alkanes include, for example, C₁₀-C₂₀ alkanes, such as C₁₂-C₁₆ alkanes such as isohexadecane or isododecane; or isoparaffins.

According to certain embodiments, the non-polar oil is an olefin such as a polyolefin. Suitable polyolefins include oligomeric or polymeric compounds such as squalene, hydrogenated polyisobutene, hydrogenated polydecene, hydrogenated poly(6-14) olefin, and polybutene.

According to certain embodiments, the non-polar oil may be present in a total concentration by weight from about 5%, 10%, 15%, 20%, 25%, 30% or 35% to about 50%, 55%, 65% or 70% by weight.

Polar Oils

In certain embodiments, the polar oil has a partition coefficient (log P) value of less than or equal to about 20 such as less than about 17, such as less than about 15.

In some embodiments, the polar oil may have a molecular weight of less than about 650 g/mol, such as less than about 500 g/mol, such as from about 150 g/mol to about 650 g/mol, such as from about 150 g/mol to about 500 g/mol. Such materials may sometimes be referred to as “mid-molecular weight polar oils.”

In some embodiments, the at least one polar oil may be a fatty ester. Suitable fatty esters include vegetable oils (glyceryl esters of fatty acids, monoglycerides, diglycerides, triglycerides) and synthetic esters.

Specific non-limiting examples of polar oils include, without limitation, Isodecyl neopentanoate (log P=6.00), Diethylhexyl malate (log P=6.61); Isopropyl myristate (log P=7.43); Isopropyl palmitate (log P=8.49); Tricaprylin (log P=9.33); Isopropyl isostearate (log P=9.37); Isostearyl neopentanoate (log P=10.25); Octyldodecyl neopentanoate (log P=11.32); Ethylhexyl palmitate (log P=11.34); Ethylhexyl stearate (log P=12.40) and Cetyl palmitate (Log P=15.59); Tridecyl trimellitate (log P=16.54); and Diisostearyl malate (log P=16.87). Other examples include propylene carbonate.

According to certain embodiments, the polar oil may be present in the composition in a total concentration from about 1%, 2%, 3%, 5%, or 10%, to about 15%, 25%, 30%, or 50% by weight.

In some embodiments, the at least one non-polar oil and the at least one polar oil having a molecular weight of less than 650 g/mol should be present in concentrations by weight such that a ratio of the concentration by weight of the at least one non-polar oil to the concentration by weight of the at least one polar oil having a molecular weight of less than 650 g/mol is at least about 1:1. According to certain embodiments, this ratio is from about 1:1, 2:1. 3:1, 4:1 or 5:1 to about 6:1, 7:1, 8:1, 9:1, 10:1, 11:1, 12:1, 13:1 or 14:1. According to certain notable embodiments, this ratio is from about 1:1 to about 14:1. According to certain other embodiments, this ratio is from about 3:1 to about 14:1.

Viscous Silicones

In some embodiments, the composition may comprise a viscous silicone, which may comprise or consist of one or more dimethicones. In some embodiments, the composition may comprise one dimethicone. In some embodiments, the composition may comprise a plurality of dimethicones.

The silicones are preferably non-volatile silicones. In some embodiments, such silicones have a viscosity ranging from about greater than 5 to 800,000 cSt, such as from 20 to 200,000 cSt at 25° C. Examples of non-volatile silicones include phenyl trimethicone, or trimethylsiloxyphenyl dimethicone. Other examples include alkyl dimethicones such as cetyl dimethicone. Phenyl trimethicone can be purchased from Dow Corning Corporation under the tradename 556 Fluid. Trimethylsiloxyphenyl dimethicone can be purchased from Wacker under the tradename PDM-1000. Cetyl dimethicone, also referred to as a liquid silicone wax, may be purchased from Dow Corning as Fluid 2502, or from Evonik DeGussa under the trade names Abil Wax 9801, or 9814.

In some embodiments, the weighted average of the molecular weights of the plurality of dimethicones may be at least 1,500 g/mol. In some embodiments, the weighted average of the molecular weights of the plurality of dimethicones is from 1000, 1500, or 2000 up to 3000, 4000, 5000, 6000, 7000, 8000, 9000, 10000, or 20,000 g/mol, including all combinations and subranges thereof. In one preferred embodiment, the weighted average of the molecular weights of the plurality of dimethicones is between 2000 and 3000 g/mol.

In some embodiments, the dimethicones may include a first dimethicone having a molecular weight less than 1,500 g/mol (such as cetyl dimethicone), and a second dimethicone having a molecular weight greater than 2,000 g/mol (such as trimethylsiloxyphenyl dimethicone).

In some embodiments, the dimethicones may include a first dimethicone this is present at less than 10%, and a second dimethicone that is present at more than 15%.

In some embodiments, the dimethicones may include a first dimethicone that is a polyalkyl dimethicone, and a second dimethicone that contains at least one aryl group.

In some embodiments, the dimethicone(s) may be present in the cosmetic composition in a total amount ranging from, for example, about 20%, 22%, or 25% to about 30%, 35%, or 40% by weight of the composition.

In some embodiments, the composition is free, or substantially free, of all other silicones.

Fillers

The composition should include a plurality of fillers, and specifically, should include both a hectorite and silica silylate. Without being held to a specific theory, it is believed the combination of these specific hectorites with silica silylate results in the unexpected stabilization of these thermoplastic block copolymer/silicone systems.

The ratio of hectorite to silica silylate may vary, but the concentrations are typically on the same order of magnitude. In some embodiments, the ratio of the amount of silica silylate (in % by weight) to hectorite (in % by weight) to may be from 4:1 to 1:1. In some embodiments, the ratio is from 3:1 to 1:1, 2:1, 1.5:1, or 1.25:1. In some preferred embodiments, the ratio is 1:1.

In some embodiments, the total amount of hectorite plus silica silylate is at least 10% of the total amount of viscous silicone in the system. In some embodiments, the total amount of hectorite plus silica silylate is at least 12% of the total amount of viscous silicone in the system. In some embodiments, the total amount of hectorite plus silica silylate is at least 15% of the total amount of viscous silicone in the system.

Hectorite

In some embodiments, the composition may comprise a hectorite, such as an ammonium hectorite compound. The hectorite should be selected from disteardimonium hectorite, stearalkonium hectorite, or both. In some embodiments, a single hectorite is present. In some embodiments, both hectorites are present.

In some embodiments, the hectorite is present in the composition in a total amount of least 0.5% by weight of the composition, preferably at least 1% by weight, and more preferably at least 2% by weight. In some embodiments, the hectorite is present in the composition in a total amount of up to 2%, 2.5%, 3%, 3.5%, 4%, or 5% by weight of the composition. In some embodiments, the hectorite is present in the composition in a total amount of 1.5% to 2.5% by weight of the composition.

Silica Silylate

In some embodiments, the composition may include silica silylate. In some embodiment, the silica silylate is silylated silica (INCI name: silica silylate) aerogel particles, which are hydrophobic silica aerogel particles. Silica aerogels are porous materials obtained by replacing (by drying) the liquid component of a silica gel with air. The preparation of hydrophobic silica aerogel particles that have been surface-modified by silylation is described more fully in U.S. Pat. No. 7,470,725, incorporated by reference herein.

In some embodiments, aerogel particles of hydrophobic silica surface-modified with trimethylsilyl groups may be chosen. For example, the aerogel sold under the name VM-2260® by the company Dow Corning, the particles of which have an average size of about 1000 microns and a specific surface area per unit of mass ranging from 600 to 800 m²/g, or the aerogel sold under the name VM-2270®, also by the company Dow Corning, the particles of which have an average size ranging from 5 to 15 microns and a specific surface area per unit of mass ranging from 600 to 800 m²/g, may be chosen.

In some embodiments, the silica silylate is present in the composition in a total amount of least 0.5% by weight of the composition, preferably at least 1% by weight, and more preferably at least 2% by weight. In some embodiments, the hectorite is present in the composition in a total amount of up to 2%, 2.5%, 3%, 3.5%, 4%, or 5% by weight of the composition.

In some embodiments, the silica silylate is present in the composition in a total amount of 1.5% to 2.5% by weight of the composition.

Other Components

The compositions may include other components. In some embodiments, the compositions may include a tackifier. In some embodiments, the compositions may include a thickening agent (“thickener”), an olfactive sensorial agent, a solvent, a preservative, antioxidant, a butter, an ester, or a combination thereof. In some embodiments, the compositions may include a pigment, a pearlescent agent, or a combination thereof. In some embodiments, the composition may include a fatty compound having a melting point in the range of 30° C. to 50° C.

In some embodiments, the composition may comprise or consist of a thermoplastic block copolymer, a hydrocarbon oil, a dimethicone, a hectorite, silica silylate, a tackifier, a thickener, an olfactive sensorial agent, a solvent, a preservative, an antioxidant, a fatty compound having a melting point in the range of 30° C. to 50° C., a colorant (such as a pigment or pearlescent agent), or a combination thereof, and may be free or substantially free of all other materials.

Tackifier

In some embodiments, the composition may include a “tackifying” resin, referred to as a tackifier. Such resins are described in particular in the Handbook of Pressure Sensitive Adhesive, edited by Donatas Satas, 3rd ed., 1989, pp. 609-619. The resin of the composition according to the invention is chosen from rosin, rosin derivatives, hydrocarbon resins and their mixtures. Preferably, the resin is an indene hydrocarbon resin which can optionally be hydrogenated.

Rosin is a mixture predominantly comprising organic acids known as rosin acids (mainly acids of abietic type and of pimaric type). Three types of rosin exist: the rosin (“gum rosin”) obtained by incision on living trees, wood rosin, which is extracted from pine stumps or wood, and tall oil (tall oil rosin), which is obtained from a by-product originating from paper manufacture.

The rosin derivatives can result in particular from the polymerization, hydrogenation and/or esterification (for example with polyhydric alcohols, such as ethylene glycol, glycerol or pentaerythritol) of rosin acids. Mention may be made, for example, of the rosin esters sold under the references Foral 85, Pentalyn H and Staybelite Ester 10 by Hercules; Sylvatac 95 and Zonester 85 by Arizona Chemical; or Unirez 3013 by Union Camp.

The hydrocarbon resins are chosen from polymers of low molecular weight which can be classified, according to the type of monomer which they comprise, into:
indene hydrocarbon resins, such as in particular the resins resulting from the polymerization predominantly of indene monomer with a minor proportion of monomer chosen from styrene, methylindene, methylstyrene and their mixtures, it being possible for these resins optionally to be hydrogenated. These resins can exhibit a molecular weight ranging from 290 to 1150 g/mol. Mention may be made, as examples of indene resins, of those sold under the references Escorez 7105 by Exxon Chem., Nevchem 100 and Nevex 100 by Neville Chem., Norsolene S105 by Sartomer, Picco 6100 by Hercules and Resinall by Resinall Corp., or the hydrogenated indene/methylstyrene/styrene copolymers sold under the “Regalite” name by Eastman Chemical, in particular Regalite R 1100, Regalite R 1090, Regalite R-7100, Regalite R1010 Hydrocarbon Resin or Regalite R1125 Hydrocarbon Resin.

In some embodiments, the tackifier may be present in a total amount from 1%, 3%, or 5% to about 10%, 12%, or 15% by weight of the composition.

Thickener

The composition may include a thickener/oil phase structuring agent chosen from among polymers. In some embodiments, the thickener may be a C10 to C30 and preferably C14 to C22 alkyl (meth)acrylate homopolymers; and more particularly, poly(stearyl acrylate) or poly(behenyl acrylate) homopolymers, such as those marketed under the trade names Intelimer® IPA 13-1 NG polymer, Intelimer® IPA 13-6 by the Air Product and Chemicals company (INCI name: POLY C10-30 ALKYL ACRYLATE). Poly(stearyl acrylate) homopolymers will be preferably be used.

In some embodiments, the thickener may be present in a total amount from 0.1%, 0.25%, or 0.4% to 0.6%, 0.75%, 1%, or 2% by weight of the composition.

Olfactive Sensorial Agent

In some embodiments, the compositions may include an olfactive sensorial agent, such as a fragrance or a flavorant. In some embodiments, the olfactive sensorial agent may comprise a refreshing agent, such as menthol and derivatives thereof, that are capable of providing, e.g., a “refreshing” or “cooling” feeling or sensation.

In some embodiments, the olfactive sensorial agent may be present in a total amount from 0.1%, 0.25%, or 0.4% to 0.6%, 0.75%, 1%, or 2% by weight of the composition.

Solvent

In some embodiments, the composition may include a solvent in addition to the hydrocarbon oils. In some embodiments, the solvent is soluble in the hydrocarbon oils. In some embodiments, the solvent may comprise or consist of glycerin, ethylhexyl glycerin and/or pentaerythrityl tetraisostearate.

In some embodiments, the solvent may be present in a total amount from 0.1%, 0.25%, or 0.4% to 0.6%, 0.75%, 1%, or 2% by weight of the composition.

Preservative and Antioxidants

In some embodiments, the composition may include a preservative and/or an antioxidant.

In some embodiments, the preservative may be present in a total amount from 0.1%, 0.25%, or 0.4% to 0.6%, 0.75%, 1%, or 2% by weight of the composition. In some embodiments, the antioxidant may be present in a total amount less than 2%, less than 1%, less than 0.5%, less than 0.25%, or less than 0.1% by weight of the composition.

Butters and Low Melt Point Fatty Compounds

In some embodiments, the composition may include a butter. Non-limiting examples of such butters include mango butter, such as that sold under the reference Lipex 203 by AarhusKarlshamn; shea butter, in particular that having the INCI name Butyrospermum Parkii Butter, such as that sold under the reference Sheasoft® by AarhusKarlshamn; cocoa butter, in particular that which is sold under the name CT Cocoa Butter Deodorized by Dutch Cocoa BV or that which is sold under the name Beurre De Cacao NCB HD703 758 by Barry Callebaut; shorea butter, in particular that which is sold under the name Dub Shorea T by Stearinerie Dubois; and their mixtures. In some embodiments, the butter may be present in a total amount from 3%, 4%, 5%, 6%, 7%, 8%, or 10% to 5%, 8%, 10%, 15%, 20%, or 30% by weight of the composition.

In some embodiments, the composition may include a fatty compound having a melting point in the range of 30° C. to 50° C. Such fatty compounds may include a butter (e.g., shea butter) or an ester, such as a glycerol oligomer ester. The glycerol oligomer esters may be chosen from diglycerol esters, particularly adipic acid and glycerol condensates, for which part of the hydroxyl groups of the glycerols have reacted with a mixture of fatty acids such as stearic acid, capric, stearic acid and isostearic acid and 12-hydroxystearic acid, preferably such as bis-diglyceryl polyacyladipate-2, for example sold under the brand Softisan 649 by Sasol.

In some embodiments, the fatty compound having a melting point in the range of 30° C. to 50° C. may be present in a total amount from 8%, 10%, 12%, or 14% to 15%, 17%, 20%, 25%, or 30%, by weight of the composition.

Colorants

In some embodiments, the composition may include a colorant. In some embodiments, the colorant is a pigment, a pearlescent agent, or a combination thereof,

In some embodiments, the composition can advantageously comprise at least one colorant chosen from pigments and/or pearlescent agents.

Pigments

As used herein, the term “pigment” refers to white or colored and inorganic (mineral) or organic particles which are insoluble in the lipophilic phase(s) and which are intended to color and/or opacify the composition and/or the deposited layer produced with the composition.

The pigments may be chosen from mineral pigments, organic pigments and composite pigments (i.e., pigments based on mineral and/or organic materials).

The pigments can be chosen from mineral pigments, in particular monochromatic pigments, organic lakes, pearlescent agents and goniochromatic pigments.

If the composition comprises them, their content varies from 0.1% to 15% by weight, with respect to the weight of the composition, and preferably from 0.5% to 12% by weight, with respect to the weight of the composition.

The mineral pigments can be chosen from metal oxide pigments, chromium oxides, iron oxides (black, yellow, red), titanium dioxide, zinc oxides, cerium oxides, zirconium oxides, chromium hydrate, manganese violet, Prussian blue, ultramarine blue, ferric blue, metal powders, such as aluminum powders or copper powder, and their mixtures.

Organic lakes are organic pigments, formed of a dye attached to a substrate. The lakes, which are also known as organic pigments, can be chosen from the materials below and their mixtures:
cochineal carmine;
organic pigments of azo dyes, anthraquinone dyes, indigoid dyes, xanthene dyes, pyrene dyes, quinoline dyes, triphenylmethane dyes or fluoran dyes.

Mention may in particular be made, among the organic pigments, of those known under the following names: D&C Blue No. 4, D&C Brown No. 1, D&C Green No. 5, D&C Green No. 6, D&C Orange No. 4, D&C Orange No. 5, D&C Orange No. 10, D&C Orange No. 11, D&C Red No. 6, D&C Red No. 7, D&C Red No. 17, D&C Red No. 21, D&C Red No. 22, D&C Red No. 27, D&C Red No. 28, D&C Red No. 30, D&C Red No. 31, D&C Red No. 33, D&C Red No. 34, D&C Red No. 36, D&C Violet No. 2, D&C Yellow No. 7, D&C Yellow No. 8, D&C Yellow No. 10, D&C Yellow No. 11, FD&C Blue No. 1, FD&C Green No. 3, FD&C Red No. 40, FD&C Yellow No. 5 or FD&C Yellow No. 6;

the organic lakes can be insoluble sodium, potassium, calcium, barium, aluminum, zirconium, strontium or titanium salts of acid dyes, such as azo, anthraquinone, indigoid, xanthene, pyrene, quinoline, triphenylmethane or fluoran dyes, these dyes possibly comprising at least one carboxylic or sulfonic acid group.

The organic lakes can also be supported by an organic support, such as rosin or aluminum benzoate, for example.

Mention may in particular be made, among the organic lakes, of those known under the following names: D&C Red No. 2 Aluminum lake, D&C Red No. 3 Aluminum lake, D&C Red No. 4 Aluminum lake, D&C Red No. 6 Aluminum lake, D&C Red No. 6 Barium lake, D&C Red No. 6 Barium/Strontium lake, D&C Red No. 6 Strontium lake, D&C Red No. 6 Potassium lake, D&C Red No. 7 Aluminum lake, D&C Red No. 7 Barium lake, D&C Red No. 7 Calcium lake, D&C Red No. 7 Calcium/Strontium lake, D&C Red No. 7 Zirconium lake, D&C Red No. 8 Sodium lake, D&C Red No. 9 Aluminum lake, D&C Red No. 9 Barium lake, D&C Red No. 9 Barium/Strontium lake, D&C Red No. 9 Zirconium lake, D&C Red No. 10 Sodium lake, D&C Red No. 19 Aluminum lake, D&C Red No. 19 Barium lake, D&C Red No. 19 Zirconium lake, D&C Red No. 21 Aluminum lake, D&C Red No. 21 Zirconium lake, D&C Red No. 22 Aluminum lake, D&C Red No. 27 Aluminum lake, D&C Red No. 27 Aluminum/Titanium/Zirconium lake, D&C Red No. 27 Barium lake, D&C Red No. 27 Calcium lake, D&C Red No. 27 Zirconium lake, D&C Red No. 28 Aluminum lake, D&C Red No. 30 lake, D&C Red No. 31 Calcium lake, D&C Red No. 33 Aluminum lake, D&C Red No. 34 Calcium lake, D&C Red No. 36 lake, D&C Red No. 40 Aluminum lake, D&C Blue No. 1 Aluminum lake, D&C Green No. 3 Aluminum lake, D&C Orange No. 4 Aluminum lake, D&C Orange No. 5 Aluminum lake, D&C Orange No. 5 Zirconium lake, D&C Orange No. 10 Aluminum lake, D&C Orange No. 17 Barium lake, D&C Yellow No. 5 Aluminum lake, D&C Yellow No. 5 Zirconium lake, D&C Yellow No. 6 Aluminum lake, D&C Yellow No. 7 Zirconium lake, D&C Yellow No. 10 Aluminum lake, FD&C Blue No. 1 Aluminum lake, FD&C Red No. 4 Aluminum lake, FD&C Red No. 40 Aluminum lake, FD&C Yellow No. 5 Aluminum lake or FD&C Yellow No. 6 Aluminum lake.

Mention may also be made of liposoluble dyes, such as, for example, Sudan Red, DC Red 17, DC Green 6, β-carotene, soybean oil, Sudan Brown, DC Yellow 11, DC Violet 2, DC Orange 5 or quinoline yellow.

The chemical substances corresponding to each of the organic colourants cited above are mentioned in the publication “International Cosmetic Ingredient Dictionary and Handbook”, 1997 edition, pages 371 to 386 and 524 to 528, published by The Cosmetic, Toiletries and Fragrance Association, the content of which is incorporated into the present patent application by reference.

The pigments may also have been subjected to a hydrophobic treatment.

The hydrophobic treatment agent can be chosen from silicones, such as methicones, dimethicones, alkoxysilanes and perfluoroalkylsilanes; fatty acids, such as stearic acid; metal soaps, such as aluminum dimyristate, the aluminum salt of hydrogenated tallow glutamate, perfluoroalkyl phosphates, perfluoroalkyl silanes, perfluoroalkyl silazanes, poly(hexafluoropropylene oxide)s, polyorganosiloxanes comprising perfluoroalkyl perfluoropolyether groups and amino acids; N-acylated amino acids or their salts; lecithin, isopropyl triisostearyl titanate, and their mixtures.

The N-acylated amino acids can comprise an acyl group having from 8 to 22 carbon atoms, such as, for example, a 2-ethylhexanoyl, caproyl, lauroyl, myristoyl, palmitoyl, stearoyl or cocoyl group. The salts of these compounds can be aluminum, magnesium, calcium, zirconium, zinc, sodium or potassium salts. The amino acid can, for example, be lysine, glutamic acid or alanine.

The term “alkyl” cited in the abovementioned compounds denotes in particular an alkyl group having from 1 to 30 carbon atoms and preferably having from 5 to 16 carbon atoms.

Hydrophobic treated pigments are described in particular in Application EP-A-1 086 683.

Pearlescent Agents

As used herein, the term “pearlescent agent” refers to colored particles of any shape, which are or are not iridescent, and which may be of natural or synthetic origin, and which exhibit a color effect via optical interference.

Mention may be made, as examples of pearlescent agents, of pearlescent pigments, such as titanium oxide-coated mica covered with an iron oxide, mica covered with bismuth oxychloride, titanium oxide-coated mica covered with chromium oxide, titanium oxide-coated mica covered with an organic dye, in particular of the abovementioned type, and also pearlescent pigments based on bismuth oxychloride.

They can also be mica particles, at the surface of which are superimposed at least two successive layers of metal oxides and/or of organic colorants.

The pearlescent agents can more particularly have a yellow, pink, red, bronze, orangey, brown, gold and/or coppery color or glint.

Mention may be made, by way of illustration of the pearlescent agents which can be introduced as interference pigment into the first composition, of gold-colored pearlescent agents sold in particular by BASF under the name Brilliant Gold 212G (Timica), Gold 222C (Cloisonne), Sparkle Gold (Timica) and Monarch Gold 233X (Cloisonne); bronze pearlescent agents sold in particular by Merck under the names Bronze Fine (17384) (Colorona) and Bronze (17353) (Colorona) and by BASF under the name Super Bronze (Cloisonne); orange pearlescent agents sold in particular by BASF under the name Orange 363C (Cloisonne) and by Merck under the names Passion Orange (Colorona) and Matte Orange (17449) (Microna); brown-colored pearlescent agents sold in particular by BASF under the names Nu-Antique Copper 340XB (Cloisonne) and Brown CL4509 (Chroma-lite); pearlescent agents with a copper glint sold in particular by BASF under the name Copper 340A (Timica); pearlescent agents with a red glint sold in particular by Merck under the name Sienna Fine (17386) (Colorona); pearlescent agents with a yellow glint sold in particular by BASF under the name Yellow (4502) (Chroma-lite); red-colored pearlescent agents with a gold glint sold in particular by BASF under the name Sunstone G012 (Gemtone); pink pearlescent agents sold in particular by BASF under the name Tan Opal G005 (Gemtone); black pearlescent agents with a gold glint sold in particular by BASF under the name Nu-Antique Bronze 240 AB (Timica); blue pearlescent agents sold in particular by Merck under the name Matte Blue (17433) (Microna); white pearlescent agents with a silvery glint sold in particular by Merck under the name Xirona Silver; and golden green pinkish orangey pearlescent agents sold in particular by Merck under the name Indian Summer (Xirona); and their mixtures.

Goniochromatic Pigments

As used herein, the term “goniochromatic pigment” refers to a pigment which makes it possible to obtain, when the composition is spread over a substrate, a color distance in the a*b* plane of the CIE 1976 colorimetric space which corresponds to a variation Dh° in the angle of hue h° of at least 20° when the angle of observation is varied with respect to the normal by between 0° and 80°, for an angle of incidence of the light of 45°.

The color distance can be measured, for example, using a spectrogonioreflectometer of the Instrument Systems brand and with the GON 360 Goniometer reference, after the composition has been spread in the fluid state with a thickness of 300 μm using an automatic spreader over a contrast chart of the Erichsen brand and with the Type 24/5 reference, the measurement being carried out on the black background of the chart.

The goniochromatic pigment can be chosen, for example, from multilayer interference structures and liquid crystal coloring agents.

In the case of a multilayer structure, the latter can comprise, for example, at least two layers, each layer being produced, for example, from at least one material chosen from the group consisting of the following materials: MgF2, CeF3, ZnS, ZnSe, Si, SiO2, Ge, Te, Fe2O3, Pt, Va, Al2O3, MgO, Y2O3, S2O3, SiO, HfO2, ZrO2, CeO2, Nb2O5, Ta2O5, TiO2, Ag, Al, Au, Cu, Rb, Ti, Ta, W, Zn, MoS2, cryolite, alloys, polymers and their combinations.

The multilayer structure may or may not exhibit, with respect to a central layer, a symmetry with regard to the chemical nature of the stacked layers.

Different effects are obtained according to the thickness and the nature of the various layers.

Examples of symmetrical multilayer interference structures are, for example, the following structures: Fe₂O₃/SiO₂/Fe₂O₃/SiO₂/Fe₂O₃, a pigment having this structure being sold under the name Sicopearl by BASF; MoS2/SiO2/mica-oxide/SiO2/MoS2; Fe2O3/SiO2/mica-oxide/SiO2/Fe2O3; TiO2/SiO2/TiO2 and TiO2/Al2O3/TiO2, pigments having these structures being sold under the name Xirona by Merck.

The liquid crystal coloring agents comprise, for example, silicones or cellulose ethers to which mesomorphic groups are grafted. Use may be made, as liquid crystal goniochromatic particles, for example, of those sold by Chenix and of those sold under the name Helicone® HC by Wacker.

Use may also be made, as goniochromatic pigment, of certain pearlescent agents, effect pigments on a synthetic substrate, in particular a substrate of alumina, silica, borosilicate, iron oxide or aluminum type, or interference glitter resulting from a polyterephthalate film.

Mention may in particular be made, as non-limiting examples of goniochromatic pigments, alone or as mixtures, of the goniochromatic pigments SunShine® sold by Sun Chemical, Cosmicolor Celeste® from Toyo Aluminum K.K., Xirona® from Merck and Reflecks Multidimensions® from BASF.

The composition according to the invention may also comprise one or more dyes.

Among the liposoluble dyes, mention may be made especially of fluoran dyes such as, for example, red Sudan, FDC Red 4, DC Red 17, Red 21, Red 27, DC Green 6, Sudan brown, Yellow 10, DC Yellow 11, DC Violet 2, DC Orange 4, DC Orange 5, Yellow quinoline, or mixtures thereof.

In some embodiments, the composition is preferably selected so as to provide a particular rheological profile. In particular, a storage modulus of the composition at an elevated temperature of 55° C. divided by a storage modulus at a temperature of 25° C. may be greater than or equal to 0.07, preferably at least 0.08, more preferably at least 0.09, still more preferably at least 0.10, and most preferably at least 0.15.

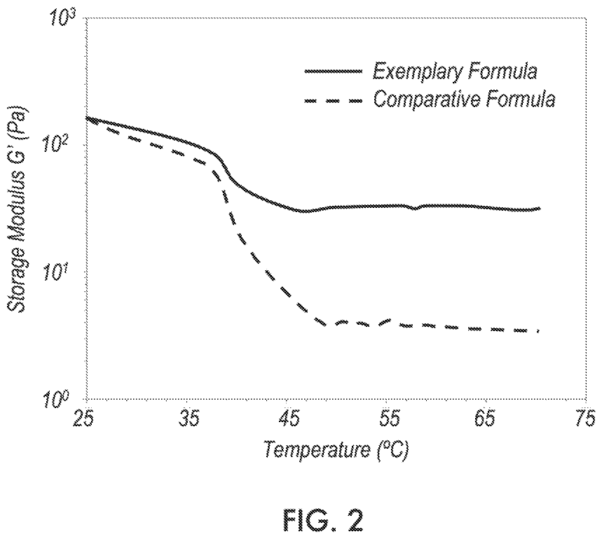

This can be seen with reference to FIG. 2. In FIG. 2, two near-identical formulas were tested. A first was an exemplary formula that includes 2% disteardimonium hectorite and 2% silica silylate, with the remainder being a base comprised of 20-30% of a combination of two dimethicones, 8-12% of a thermoplastic block copolymer, 35-40% of a combination of hydrocarbon oils, and the remainder various other components as disclosed herein. A second was a comparative formula, where the disteardimonium hectorite was replaced with a different filler, lauroyl lysine. As seen in FIG. 2, the storage modulus of the exemplary composition at 55° C. remains relatively high as compared to the modulus for the comparative formula using lauroyl lysine. Specifically, in this example, the resulting quotient, sometimes referred to as G′_{DECAY}, from dividing the storage modulus at 55° C. by the storage modulus at 25° C., is 0.196 for the exemplary formula, and 0.0248 for the comparative formula.

Test Method: After producing the compositions, the modulus values were determined at 25° C., with a controlled-stress rheometer (e.g., a Haake RS 150) equipped with a sand-blasted titanium body fitted with an anti-evaporation device and a sandblasted 40 mm/0° parallel plate measuring body with the following conditions: no preshear, 30 s temperature equilibration at 25° C., temperature ramp from 25° C. to 70° C., 3° C./min., applied strain 0.2% at ω=1.0 s⁻¹.

With the values in hand, G′_{DECAY} can be calculated by dividing the value at an elevated temperature (such as 55° C. is then divided by to the temperature at 25° C.). Note that in some embodiments, this can be represented as a percentage.

In some embodiments, an alternative approach can be to divide the storage modulus (G′) at an elevated temperature by the storage modulus of the composition at the initial point, then multiplying by 100, to get a “relative” value as a percent change, sometimes referred to as G′REL.

### Example 1

The compositions listed in Table 1 were produced by combining the components at 90-95° C., mixing until uniform, and allowing to cool. Usages are listed in % by weight of the total composition. In some examples a silica silylate that was spherical and exhibited an oil absorbance varying from about 2 mL/g to about 12 mL/g was used (“Silica Silylate 1”). Due to the wide range of performance, this may sometimes be referred to as a “low oil absorbing” silica silylate. In some examples, a silica silylate with irregular shaped particles and exhibited an oil absorbance of at least about 6 mL/g was used (“Silica Silylate 2”), e.g., a consistently “high oil absorbing” silica silylate. The dimethicones included cetyl dimethicone and trimethylsiloxyphenyl dimethicone at approximately a 1:4 ratio. The hydrocarbon oils included three polar oils and two non-polar oils at about a 1:1.15 ratio, including esters, and a saturated or unsaturated polyolefin. The other materials include two low melting temperature fatty compounds at a total amount of over 10%, solvents, preservative, a tackifier, and a thickener.

TABLE 1

| Material | Ex-1 | Ex-2 | Ex-3 | C-1 | C-2 | C-3 | C-4 | C-5 |
|---|---|---|---|---|---|---|---|---|
| Thermoplastic | 8-12 | 8-12 | 8-12 | 8-12 | 8-12 | 8-12 | 8-12 | 8-12 |
| Block | | | | | | | | |
| Copolymer | | | | | | | | |
| Dimethicone(s) | 20-30 | 20-30 | 20-30 | 20-30 | 20-30 | 20-30 | 20-30 | 20-30 |
| Hydrocarbon | 37-38 | 37-38 | 37-38 | 37-38 | 37-38 | 37-38 | 39-40 | 38.5-39.5 |
| Oil(s) | | | | | | | | |
| Silica Silylate 1 | 2 | 2 | — | 2 | 2 | 2 | — | 2 |
| Silica Silylate 2 | — | — | 2 | — | — | — | — | — |
| Stearalkonium | — | 2 | — | — | — | — | — | — |
| Hectorite | | | | | | | | |
| Disteardimonium | 2 | — | 0.5 | — | — | — | 2 | 0.5 |
| Hectorite | | | | | | | | |
| Lauroyl Lysine | — | — | — | 2 | — | — | — | — |
| Kaolin | — | — | — | — | 2 | — | — | — |
| Fumed Silica | — | — | — | — | — | 2 | — | — |
| Pigments and | 0.1-2 | 0.1-2 | 0.1-2 | 0.1-2 | 0.1-2 | 0.1-2 | 0.1-2 | 0.1-2 |
| Pearlescents | | | | | | | | |
| Other Materials | q.s. | q.s. | q.s. | q.s. | q.s. | q.s. | q.s. | q.s. |
| Disclosed Herein | | | | | | | | |

Various embodiments of the present disclosure as shown as exemplary formulas (Ex-1, Ex-2, and Ex-3), along with comparative formulas (C-1, C-2, C-3, C-4, and C-5).

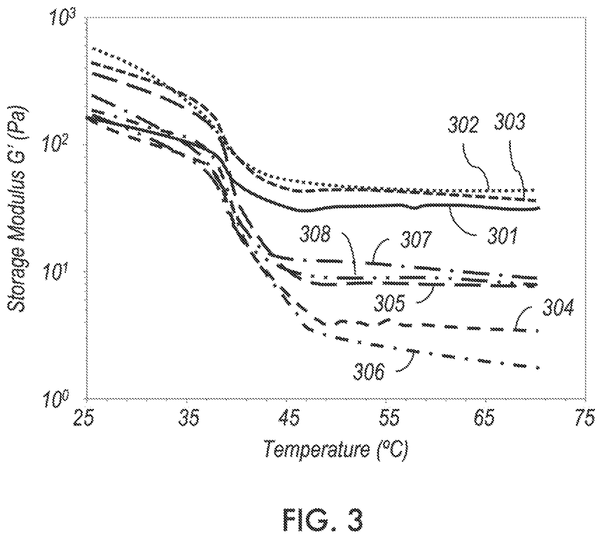

The above formulas were compared using the rheological test method disclosed herein relating to comparing a storage modulus at 55° C. to a storage modulus at 25° C. FIG. 3 shows the results in graphical form, where storage modulus values for all eight compositions are shown: Ex-1 (ref 301, solid line), Ex-2 (ref 302, dotted line), Ex-1 (ref 303, short dashed line), C-1 (ref 304, medium dashed line), C-2 (ref 305, long dashed line), C-3 (ref 306, medium dash and dotted line), C-4 (red 307, long dash and dotted line), and C-5 (ref 308, long dash and double dotted line). The G′_{DECAY} values calculated from these are summarized in Table 2, below.

The formulas were also placed into a stability chamber maintained at a temperature of a 55° C. for one week, after which the samples were visually evaluated for settling/syneresis. The results are summarized in Table 2, below.

TABLE 2

| Test | Ex-1 | Ex-2 | Ex-3 | C-1 | C-2 | C-3 | C-4 | C-5 |
|---|---|---|---|---|---|---|---|---|
| G′_{DECAY} | 0.196 | 0.0810 | 0.0986 | 0.0248 | 0.0222 | 0.0130 | 0.0498 | 0.0579 |
| Visual Settling | No | No | Minor | Severe | Severe | Severe | Severe | Severe |

As is seen, the use of both the disclosed hectorites, as well as the claimed amount of silica silylate surprisingly results in stable formulas. There is a sharp divide in G′_{DECAY}, between those with severe settling/syneresis issues and those with improved performance. As is seen, embodiments of the composition have G′_{DECAY} values ≥0.08. In this example, the values range from 0.08-0.2, and all show substantial improvement in settling/syneresis as compared to the other comparative formulas with G′_{DECAY} less than 0.08.

It is surprising that the use of other fillers as replacements for the hectorites, does not work. For example, replacing the disteardimonium hectorite with any other filler material, such as fumed silica (C-3), kaolin (C-2), or lauroyl lysine (C-1), results in severe settling/syneresis issues. Removal of the silica silylate (C-4) results also results in severe settling/syneresis issues.

Without being bound by any theory, it is of note that a usage level of 0.5% by weight of the hectorite may sometimes be insufficient, depending on the particle shape and/or the oil absorbance capability of the silica silylate. When using 0.5% of a hectorite and 2% of a silica silylate with a lower oil uptake (C-5), the formula is still unstable with severe settling/syneresis issues, while 0.5% of the hectorite and 2% of a silica silylate with a consistently higher oil update (Ex-3), the formula is borderline stable, with only minor settling/syneresis issues.

Also disclosed is a method for providing a stable, high gloss, low tack lip composition. The method uses a composition as disclosed herein. The method includes forming a double phase product on the lips from a single application of a product by applying a composition as disclosed herein, breaking the composition such that it forms a first layer comprising the thermoplastic block copolymer in contact with the lips and a second layer comprising the dimethicone on top of the first layer.

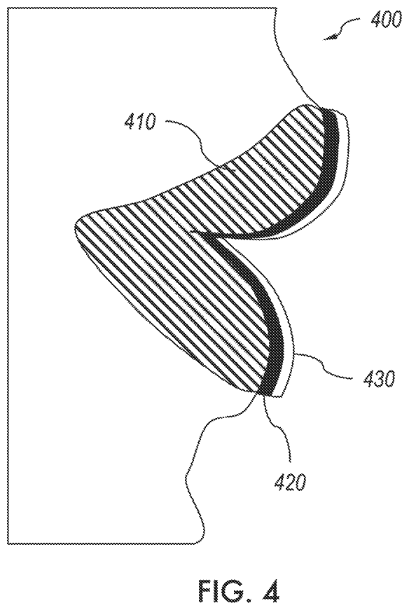

As seen in FIG. 4, when applied to the lips 410 of a person 400, the product forms a first layer 420 in contact with the lips 410, and a second layer 430 in contact with the first layer 410.

Those skilled in the art will recognize or be able to ascertain using no more than routine experimentation, many equivalents to the specific embodiments of the invention described herein. Such equivalents are intended to be encompassed by the following claims.

What is claimed is:

1. A composition for application to lips, comprising:
a first phase comprising:
at least about 8% by weight of a thermoplastic block copolymer comprising styrenated blocks; and
at least one hydrocarbon oil; and
a second phase comprising at least 20% of a dimethicone;
a hectorite selected from disteardimonium hectorite, stearalkonium hectorite, or both; and
silica silylate.

2. The composition according to claim 1, wherein the dimethicone comprises a plurality of dimethicones.

3. The composition according to claim 2, wherein the plurality of dimethicones includes a first dimethicone having a molecular weight less than 1,500 g/mol, and a second dimethicone having a molecular weight greater than 2,000 g/mol.

4. The composition according to claim 3, wherein the first dimethicone is a cetyl dimethicone and the second dimethicone is a trimethylsiloxyphenyl dimethicone.

5. The composition according to claim 2, wherein the plurality of dimethicones includes a first dimethicone that is present at less than 10%, and a second dimethicone that is present at more than 15%.

6. The composition according to claim 5, wherein the weighted average of the molecular weights of the plurality of dimethicones is at least 2,000 g/mol.

7. The composition according to claim 2, wherein the plurality of dimethicones includes a first dimethicone that is a polyalkyl dimethicone, and a second dimethicone that contains at least one aryl group.

8. The composition according to claim 1, wherein the first phase comprises at least about 10% by weight of the thermoplastic block copolymer.

9. The composition according to claim 1, wherein the composition comprises at least 0.5% by weight of the hectorite.

10. The composition according to claim 9, wherein the composition comprises at least 1% by weight of the hectorite.

11. The composition according to claim 10, wherein the composition comprises 1.5% to 2.5% by weight of the hectorite.

12. The composition according to claim 1, wherein the hydrocarbon oil comprises a saturated or unsaturated polyolefin.

13. The composition according to claim 12, wherein the saturated or unsaturated polyolefin is hydrogenated polyisobutene.

14. The composition according to claim 1, wherein a storage modulus at a temperature of 55° C. divided by a storage modulus at a temperature of 25° C. is greater than or equal to 0.08.

15. The composition according to claim 1, where a ratio of the amount of silica silylate to the amount of hectorite is between 4:1 and 1:1.

16. The composition according to claim 1, further comprising a pigment, a pearlescent agent, or a combination thereof.

17. The composition according to claim 16, further comprising a thickener, an olfactive sensorial agent, a solvent, a preservative, a butter, an ester, or a combination thereof.

18. The composition according to claim 16, further comprising a fatty compound having a melting point in the range of 30° C. to 50° C.

19. The composition according to claim 1, wherein the second phase is dispersed in the first phase.

20. A method for providing a high gloss, low tack lip composition, comprising:
forming a double phase product on the lips from a single product by applying a composition according to claim 1 and breaking the composition such that it forms a first layer comprising the thermoplastic block copolymer in contact with the lips and a second layer comprising the dimethicone on top of the first layer.
